(19) United States
(12) Patent Application Publication
(10) Pub. No.: US 2026/0112475 A1
(43) Pub. Date: Apr. 23, 2026

(54) Cognitive Training System and Method using Mnemonic Story and Game Experience

(71) Applicant: KNOW SYSTEMS CORP, MIDDLETOWN, DE (US)

(72) Inventors: Michael E. Kasaba, Malibu, CA (US); Russell Clarence Lakey, Culver City, CA (US)

(21) Appl. No.: 18/918,277

(22) Filed: Oct. 17, 2024

(51) Int. Cl. G16H 20/70; G06F 3/16; G06F 40/205

(52) U.S. Cl. CPC G16H 20/70; G06F 3/167; G06F 40/205

(57) ABSTRACT

A system and method for a cognitive training system is described. The cognitive training method includes generating a standalone sentence for a fact using a large language model (LLM), generating a mnemonic story based on the standalone sentence, and generating a mnemonic story image based on the mnemonic story using a generative artificial intelligence (AI) model. The method further includes assigning a place at a location to the fact based on an input received from a user through a user device in communication with the cognitive training system and rendering a cartoon illustration of the place. The method further includes generating a fact-memory aid associated with the fact that includes a fact component, a mnemonic component, and a place component. A user of the cognitive training system may participate in a game experience provided by a game engine to help remember the details of the fact-memory aid.

### BACKGROUND

The present disclosure generally relates to memory aids and cognitive training, and in particular, to a system and method for providing cognitive training using a mnemonic story and game experience.

Cognitive function broadly refers to mental processes that allow people to acquire knowledge, manipulate information, and reason. Cognitive function can include many varied and complex brain activities, such as attention, memory, processing speed, and executive functions like reasoning, planning, problem solving, and multitasking. Cognitive decline is a general term for a range of conditions that can impact a person's ability to think, learn, remember, and make decisions.

Recent research into cognitive decline and cognitive function has demonstrated the potential of using mnemonic memory exercises as a form of cognitive training to improve memory function and combat the effects of cognitive decline. In general terms, mnemonics relates to a learning technique that assists with information retention or retrieval in human memory, typically by associating facts, ideas, or phrases with something that is easier to remember, such as a rhyme or an acronym. Mnemonics can be used to help store information in both short-term and long-term memory.

There is a need in the art for a system and method that provides cognitive training using a mnemonic story and game experience to assist users with acquiring and retaining knowledge.

### SUMMARY

A system and method for providing cognitive training using a mnemonic story and game experience is described herein.

In one aspect, a method of providing cognitive training for a user implemented by a cognitive training system including at least one processor is provided. The method includes generating a standalone sentence for a fact using a large language model (LLM) of the cognitive training system. The method also includes generating a mnemonic story based on the standalone sentence using the LLM and generating a mnemonic story image based on the mnemonic story using a generative artificial intelligence (AI) model of the cognitive training system. The method further includes assigning a place at a location to the fact based on an input received from a user through a user device in communication with the cognitive training system. The method also includes rendering a cartoon illustration of the place using a cartoon rendering module of the cognitive training system. The method further includes generating, by the at least one processer, a fact-memory aid associated with the fact that includes a fact component, a mnemonic component, and a place component.

In another aspect, a cognitive training system is provided. The cognitive training system includes a computing system having at least one processor. The system also includes a database in communication with the computing system and a communication interface allowing communication with a user device associated with one or more users. The cognitive training system further includes storage for computer-readable instructions that, when executed by the at least one processer, cause the at least one processor to: generate a standalone sentence for a fact, generate a mnemonic story based on the standalone sentence, generate a mnemonic story image based on the mnemonic story, assign a place at a location to the fact, based on an input received from a user through the user device, render a cartoon illustration of the place, and generate a fact-memory aid associated with the fact that includes a fact component, a mnemonic component, and a place component.

Other systems, methods, features and advantages of the disclosure will be, or will become, apparent to one of ordinary skill in the art upon examination of the following figures and detailed description. It is intended that all such additional systems, methods, features and advantages be included within this description and this summary, be within the scope of the disclosure, and be protected by the following claims.

### BRIEF DESCRIPTION OF THE DRAWINGS

The disclosure can be better understood with reference to the following drawings and description. The components in the figures are not necessarily to scale, emphasis instead being placed upon illustrating the principles of the disclosure. Moreover, in the figures, like reference numerals designate corresponding parts throughout the different views.

FIG. 1 is a block diagram of an example embodiment of a cognitive training system;

FIG. 2 is a representative flowchart of an example embodiment of a method of providing a fact-memory aid for use with a cognitive training system;

FIG. 3 is a representative flowchart of an example embodiment of a process for generating a standalone sentence associated with a fact as part of the cognitive training system;

FIG. 4 is a representative view of an example embodiment of a fact for the cognitive training system;

FIG. 5 is a representative flowchart of an example embodiment of a process for creating a mnemonic story image associated with a fact for the cognitive training system;

FIG. 6 is a representative view of an example embodiment of a mnemonic story image associated with a fact;

FIG. 7 is a representative view of an example embodiment of an image of a place associated with a fact for the cognitive training system;

FIG. 8 is a representative flowchart of an example embodiment of a process for selecting locations and places to be used as part of the cognitive training system;

FIG. 9 is a representative view of an example embodiment of a cartoon illustration of a place associated with a fact and an audio presentation of the fact;

FIG. 10 is a representative view of components associated with a fact-memory aid to be used as part of the cognitive training system;

FIG. 11 is a representative view of an example embodiment of a fact-memory aid to be used as part of the cognitive training system;

FIG. 12 is a schematic view of a game engine of the cognitive memory system for providing a game experience to a user;

FIG. 13 is a representative flowchart of an example embodiment of a process for providing a game experience to a user as part of the cognitive memory system;

FIG. 14 is a schematic view of an example embodiment of a user interface for interacting with the game experience of the cognitive memory system;

FIG. 15 is a representative view of an example embodiment of a spaced retrieval interval for a game experience associated with a fact;

FIG. 16 is a representative view of an example embodiment of a user interface for the game experience associated with a fact;

FIG. 17 is a representative view of an example embodiment of a first game of the game experience for the cognitive memory system;

FIG. 18 is a schematic view of an example embodiment of inputs and outputs associated with the game engine for generating the game experience of the cognitive memory system;

FIG. 19 is a representative view of an example embodiment of another game of the game experience for the cognitive memory system;

FIG. 20 is a representative flowchart of an example embodiment of a method for a user-created fact-memory aid; and

FIG. 21 is a representative view of an example embodiment of a user interface for generating a user-created fact-memory aid.

### DETAILED DESCRIPTION

According to the techniques described herein, a system and method for cognitive training using a mnemonic story and game experience is provided. The example embodiments allow users to interact with fact-memory aids using mnemonic stories associated with various facts and engage in a game experience to reinforce learning and memory retention of the facts.

The example embodiments described herein make use of methods and systems employing artificial intelligence (AI). As used herein, “artificial intelligence” may include any known methods or techniques in machine learning and related fields. As examples, artificial intelligence may include systems and methods used in generative AI, natural language processing (NLP), large language models (LLMs), and similar fields.

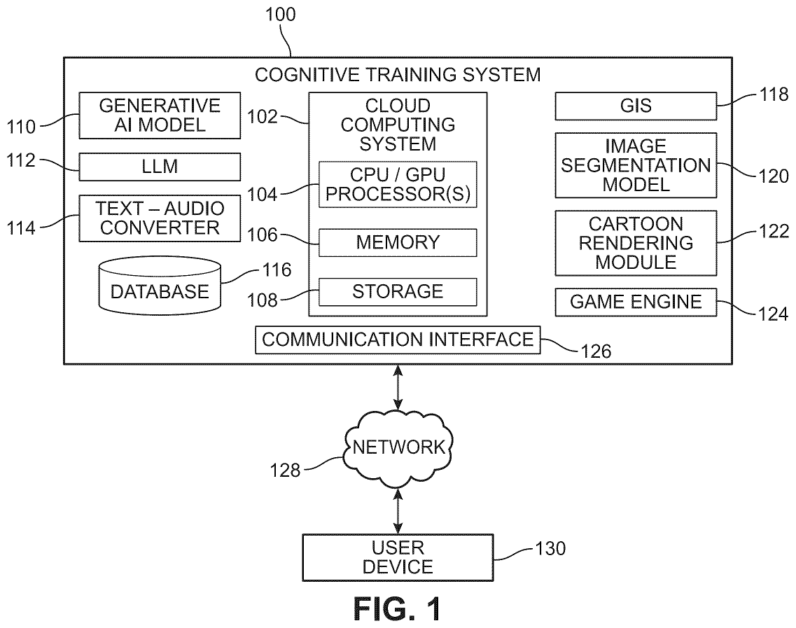

FIG. 1 is a block diagram of an example embodiment of a cognitive training system 100 (also referred to herein as “system 100”) to assist users with improving memory and general cognitive function using a mnemonic story and game experience. In some embodiments, components of system 100 may be implemented in hardware, software, and/or a combination of hardware and software to execute the functions and operations described herein to provide a cognitive training system. As will be described in more detail below, system 100 uses artificial intelligence (AI), including LLMs and generative AI models, to process and analyze a plurality of facts from source material and use those facts to generate fact-memory aids that associate each fact with a mnemonic story, a mnemonic story image and a place. The user may interact with a game engine of system 100 to participate in a game experience that assists the user with remembering and retaining the fact through recall and recognition of the components of the fact-memory aid.

In an example embodiment, cognitive training system 100 may include at least one computer system having a processor configured to execute instructions or programs to implement the techniques described herein. The term “computer system” refers to the computing resources of a single computer, the partial computing resources of a single computer, a plurality of computers communicating with one another, a network of remote servers, or other computing devices having at least one processor. In one embodiment, the computer system may be provided by a cloud computing system 102 that includes one or more processors 104, for example, central processing units (CPU) and/or graphics processing units (GPU), configured to implement the functions of cognitive training system 100 and/or components of system 100, including functions of the various modules described herein.

Cloud computing system 102 may also include a memory 106 and persistent storage 108. In an example embodiment, memory 106 and storage 108 are computer readable storage media. Memory 106 may include random access memory (RAM) and/or cache memory. In general, memory 106 may include any suitable volatile or non-volatile computer readable storage media. In some embodiments, persistent storage 108 may be a magnetic hard disk drive, a solid-state hard drive, a semiconductor storage device, read-only memory (ROM), erasable programmable read-only memory (EPROM), flash memory, or any other computer readable storage media that is capable of storing program instructions or digital information. In some embodiments, the media used by storage 108 may also be removable. For example, a removable hard drive may be used for storage 108. Other examples include optical and magnetic disks, thumb drives, and smart cards that are inserted into a drive for transfer onto another computer readable storage medium that is also part of storage 108. Instructions for executing operations of cognitive training system 100 may be stored in memory 106 and/or storage 108 for execution by processor(s) 104.

One or more programs may be stored in storage 108 for execution by one or more of the respective processors 104 via one or more memories of memory 106. For example, the one or more programs may include software instructions that, when executed by the one or more processors 104, cause cloud computing system 102 to perform the operations of cognitive training system 100 described herein and shown in connection with the accompanying Figures.

Aspects of the example embodiments are described herein with reference to flowchart illustrations and/or block diagrams of methods, apparatus (systems), and computer program products according to the embodiments. It will be understood that each block of the flowchart illustrations and/or block diagrams, and combinations of blocks in the flowchart illustrations and/or block diagrams, can be implemented by computer readable program instructions.

In some embodiments, cognitive training system 100 also includes specialized components or modules, which may be implemented in hardware, software, or a combination of hardware and software, to execute various functions associated with providing and operating cognitive training system 100. In an example embodiment, cognitive training system 100 includes a generative AI model 110 that is used to generate images from text prompts and a large language model (LLM) 112 that is used to parse text, segment and identify facts from source materials, and generate standalone sentences and associated prompts. The functions of generative AI model 110 and LLM 112 will be described in more detail below.

In some embodiments, system 100 also includes a text-audio converter 114. Text-audio converter 114 is configured to receive text inputs, such as a fact, standalone sentence, mnemonic story, keywords, etc., and convert the text inputs into audio outputs. For example, the audio outputs from text-audio converter 114 may be provided to a user through a speaker of a user device.

In some embodiments, system 100 includes at least one database 116. Database 116 is configured to store information associated with facts, mnemonic stories, mnemonic story images, place images, place illustrations, keywords, topics, etc., as well as store data and information associated with various operations and functions of system 100, such as instructions, prompts, and algorithms to implement modules described herein. In some embodiments, database 116 may also be configured to store data or information associated with one or more users of system 100. Database 116 may be co-located with system 100, may be a remote database that is accessible by cloud computing system 102 and/or other components of system 100 over a communication network, or may be a combination of local and remote databases. Database 116 may include any kind of storage devices, including but not limited magnetic, optical, magneto-optical, and/or memory, including volatile memory and non-volatile memory.

In some embodiments, cognitive training system 100 may include a geographic information system (GIS) 118. GIS 118 may be configured to capture, store, and manage spatial or geographic data, including place addresses, geographical coordinates, place images, street view images, user location information, etc. In some embodiments, GIS 118 may be further configured to generate visual representations of spatial or geographic data, such as maps, routes, place location markers or icons, etc. Functions of GIS 118 may be implemented using one or more service providers that supply geographic data, satellite or aerial images and information, traffic information, street view images, routing information, etc. to system 100.

In an example embodiment, system 100 includes an image segmentation model 120. Generally, image segmentation is a computer vision technique that partitions a digital image into discrete groups of pixels (e.g., image segments) for the purposes of object detection and related tasks. In the present embodiments, image segmentation model 120 is configured to recognize and detect buildings or façades shown in place images. In some cases, image segmentation model 120 may also score the place images to determine which images have the highest scores for building or façade presence (i.e., greatest amount of building or façade shown in the place image).

In some embodiments, system 100 includes a cartoon rendering module 122. Cartoon rendering module 122 is configured to transform a place image, such as a photograph of a building or façade, into a cartoon rendering or illustration. In some cases, cartoon rendering module 122 simplifies or exaggerates features or characteristics of a photograph to create a cartoon illustration. For example, extraneous details in a photograph may be omitted, while prominent or distinctive features may be emphasized or highlighted. Additionally, in some embodiments, cartoon rendering module 122 may be configured to generate a cartoon illustration of a place image in various artistic styles. For example, styles of cartoon illustration may include but are not limited to classic, modern, futuristic, anime, comic book, or other types of cartoon art or animation.

In some embodiments, system 100 also includes a game engine 124. Game engine 124 is configured to use facts, fact information, mnemonic stories, mnemonic story images, place information, place images, place illustrations, keywords, topics, subtopics, categories, as well as other information associated with a fact-memory aid to generate games for a user. In an example embodiment, game engine 124 may generate and implement a plurality of different games, including different types of games (i.e., games with different rules or objectives) for one or more facts or fact-memory aids. Game engine 124 may render and send for display graphics, text, and animations as part of a game experience for a user. As will be described in more detail below, game engine 124 allows a user to play games of various levels of difficulty and detail to assist the user with remembering and recalling information associated with a fact and a fact-memory aid as part of cognitive training system 100.

In addition, cloud computing system 102 may include additional computing system components, such as a bus to provide communication between processor(s) 104, memory 106, and storage 108, as well as other components of system 100, such as a communication interface 126, as well as various input/output (I/O) interface(s) that facilitate communication between the various components of system 100. Communication interface 126 provides for communications with other data processing systems or devices over a communication network 128. In an example embodiment, communication interface 126 may include one or more network interface cards. Communication interface 126 may provide communications through the use of either or both physical and wireless communications links. In an example embodiment, communication interface 126 may be configured to communicate using multiple types or formats of communication, including, but not limited to broadcast, multicast or other one-to-many communication sessions with a plurality of users, as well as one or more one-on-one communication sessions with individual users, such as two-way communication sessions that include transmitting and/or receiving video, audio, and/or text.

Communication network 128 may be implemented by any number of any type of communications network (e.g., LAN, WAN, Internet, Intranet, VPN, etc.). The computer or other processing systems of the example embodiments may include any conventional or other communications devices to communicate over the network via any conventional or other protocols. The computer or other processing systems may utilize any type of connection (e.g., wired, wireless, etc.) for access to network 128. Local communication media may be implemented by any suitable communication media (e.g., local area network (LAN), hardwire, wireless link, Intranet, etc.).

As shown in FIG. 1, a user of cognitive training system 100 may interact with system 100 through a user device 130. In different embodiments, user device 130 may take any one or more of a variety of different forms or devices. In some embodiments, user device 130 may be embodied in a computer on which the user may engage with system 100. User device 130 may also be embodied in a mobile device, such as a smartphone or tablet computer, on which the user may engage with system 100. In still another embodiment, user device 130 may be embodied in a virtual reality (VR) or augmented reality (AR) headset on which the user may engage with system 100. It should be understood that the examples of user device 130 are not limiting and other devices or forms of user device 130 may be provided to interact with system 100.

In some embodiments, user access to components and functions of cognitive training system 100 may be obtained through a website or application that acts as an intermediate interface between one or more users with user devices 130 accessing cognitive training system 100. The website or application may allow users to sign up or register with cognitive training system 100 and may allow users to browse through facts or conduct initial queries or searches to find relevant facts, as will be further described in detail below.

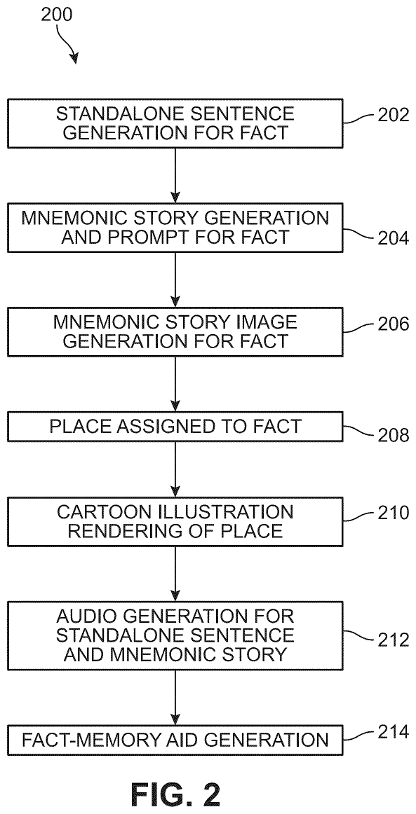

In some embodiments, a fact-memory aid is a mnemonic device for a fact that combines a standalone sentence of the fact with a mnemonic story, a mnemonic image of that story, and a place illustration associated with a location. Together these elements of the fact-memory aid help a user remember and recall the fact. Referring now to FIG. 2, a flowchart of an example embodiment of a method 200 of providing a fact-memory aid for use with a cognitive training system is shown. In an example embodiment, method 200 may be implemented by components of cognitive training system 100, described above.

In some embodiments, method 200 may begin with an operation 202 where a standalone sentence is generated for a fact. For example, in one embodiment, LLM 112 of system 100 may be used to input a fact and output a standalone sentence that contains the fact. The standalone sentence is a simple retelling or statement of the fact that includes the necessary context and information associated with that fact. Next, method 200 includes an operation 204 where a mnemonic story is generated based on the standalone sentence for the fact from operation 202. For example, at operation 204, LLM 112 of system 100 may be used to generate the mnemonic story at operation 204 based on the standalone sentence. The mnemonic story is an absurd, unique, and/or memorable phrase that may include rhyming, chunking, or wordplay carried out with the main subjects of the fact to create a mnemonic that assists a user with remembering and/or recalling the fact.

In some embodiments, operation 204 may further include generating a mnemonic story image prompt associated with the mnemonic story to provide to another component of system 100 (e.g., generative AI model 110) to generate a mnemonic story image based on the mnemonic story generated at operation 204. For example, a mnemonic story image prompt may include instructions, limitations, requirements, and/or rules for generating a mnemonic story image based on the mnemonic story for the fact. At operation 206, a mnemonic story image is generated for the fact using the mnemonic story image prompt generated at operation 204. For example, in one embodiment, the mnemonic story image prompt generated as part of operation 204 of method 200 using LLM 112 may then be provided to generative AI model 110 to execute the prompt and generate the mnemonic story image for the fact at operation 206.

Next, method 200 may proceed to an operation 208. At operation 208, a place at a location may be assigned to the fact. In some cases, a place may be a building or façade at a location near a particular user or at a location known to a user. In other cases, a place may be at a location associated with a general geographic area, such as a city, zip code, county, state, etc. In still other cases, a selection of one or more places at different locations may be specified or chosen by a user. In one embodiment, a place assigned to the fact at operation 208 may be obtained from GIS 118 of system 100, including the location of the place, such as an address or geographical coordinates.

Method 200 further includes an operation 210 where a place image from the place assigned to the fact at operation 208 is rendered as a cartoon place illustration. For example, at operation 210, a place image, such as a photograph of a building or façade, is transformed by cartoon rendering module 122 of system 100 into a cartoon rendering or illustration of the place. As described above, the cartoon place illustration generated at operation 210 typically includes simplified or exaggerated features or characteristics of the photograph of the place image that is assigned to the fact. With this arrangement, the cartoon place illustration generated at operation 210 may be more recognizable or memorable for the user than the place image.

In some embodiments, method 200 also includes an operation 212. At operation 212, audio files are generated for either or both of the standalone sentence (e.g., from operation 202) and the mnemonic story (e.g., from operation 204). For example, at operation 212, text-audio converter 114 of system 100 may be used to generate audio from the text of the standalone sentence and/or the mnemonic story associated with the fact. The audio generated at operation 212 may be output via a speaker of a user device (e.g., user device 130) to help further reinforce the standalone sentence and/or the mnemonic story in the memory of the user.

In an example embodiment, method 200 includes an operation 214. At operation 214 a fact-memory aid for a given fact is generated. For example, the fact-memory aid combines the standalone sentence for a fact (e.g., generated at operation 202) with the mnemonic story (e.g., generated at operation 204), the mnemonic image of that story (e.g., generated at operation 206), and the place illustration associated with a location (e.g., generated at operation 210). Additionally, in some cases, the audio files generated at operation 212 may also be combined with the fact-memory aid. Together these elements of the fact-memory aid generated at operation 214 of method 200 help a user remember and recall the fact.

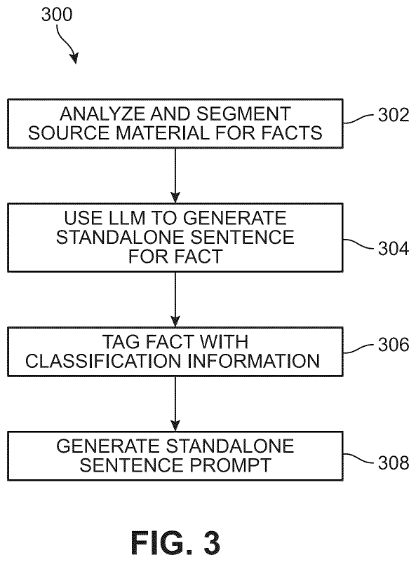

FIG. 3 is a representative flowchart of a process 300 for generating a standalone sentence associated with a fact as part of cognitive training system 100. In an example embodiment, process 300 may be implemented by components of system 100 as part of operation 202 of method 200, described above. In this embodiment, process 300 begins with an operation 302 where source material is analyzed and segmented into one or more facts. For example, the source material may be a reference publication, book, paper, lecture, website, transcript, or any other resource that includes information about any category, topic, or subtopic of knowledge, facts, statistics, or data. In one embodiment, operation 302 of process 300 may be implemented by LLM 112 of system 100 to analyze and segment the source material into one or more facts.

Next, process 300 may include an operation 304 where a large language model, such as LLM 112, is used to generate a standalone sentence for each fact derived from operation 302. For example, as described above, the standalone sentence generated at operation 304 is a simple retelling or statement of the fact that includes the necessary context and information associated with that fact. After the standalone sentence is generated, process 300 may proceed to an operation 306. At operation 306, each fact and its associated standalone sentence is tagged with relevant classification information. For example, at operation 306, a fact and its associated standalone sentence may be tagged or indexed with classification information that includes but is not limited to a subject, category, topic, subtopic, and/or keywords that relate to the fact.

In some embodiments, process 300 may further include an operation 308. At operation 308, a standalone sentence prompt may be generated to be utilized by another component of system 100 to generate a mnemonic story based on the standalone sentence. For example, in one embodiment, LLM 112 may generate a standalone sentence prompt at operation 308 that is provided as an input for generating a mnemonic story for the standalone sentence and the fact. In an example embodiment, the standalone sentence prompt may include instructions, limitations, requirements, and/or rules for generating a mnemonic story based on the standalone sentence for the fact. In some cases, the prompt may require that the mnemonic story be an absurd, unique, and/or memorable phrase that includes rhyming, chunking, or wordplay carried out with the main subjects of the fact.

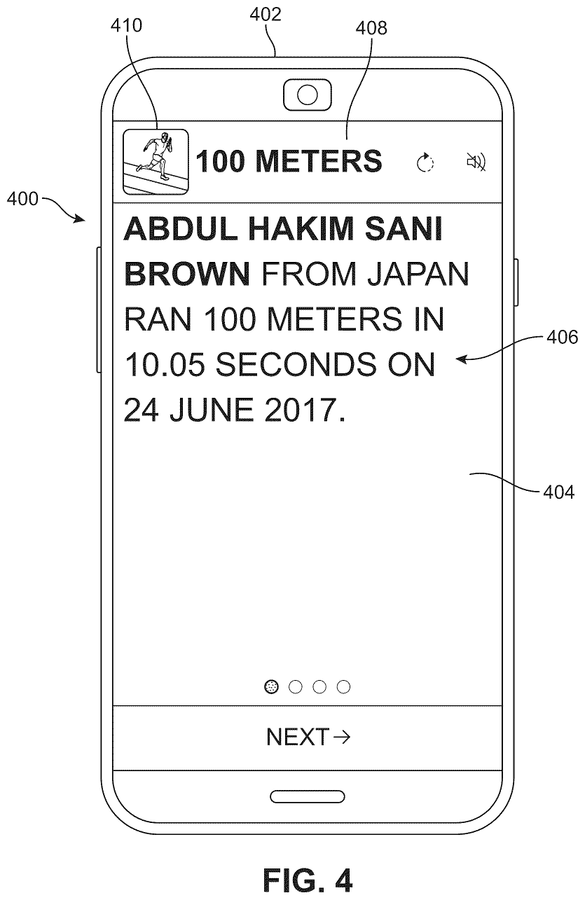

Referring now to FIG. 4, a representative view of an example embodiment of a fact 400 for cognitive training system 100 is shown. In some embodiments, fact 400 may be displayed to a user through a user device 130, as described above in reference to FIG. 1. In this embodiment, fact 400 is shown to a user on a user device in the form of a smartphone 402 through a display 404. As shown in FIG. 4, a standalone sentence 406 (e.g., “Abdul Hakim Sani Brown from Japan ran 100 meters in 10.05 seconds on 24 Jun. 2017.”) associated with fact 400 is displayed on display 404 of smartphone 402. In some embodiments, display 404 of smartphone 402 may also provide related classification information to the user related to fact 400. For example, in this embodiment, a top banner on display 404 includes a topic 408 (e.g., “100 meters”) and an icon 410 associated with a category (e.g., track and field). In other embodiments, additional classification information associated with fact 400 and/or standalone sentence 406 (e.g., tagged at operation 306 of process 300) may be provided to the user through display 404 of smartphone 402 using text and/or graphical icons.

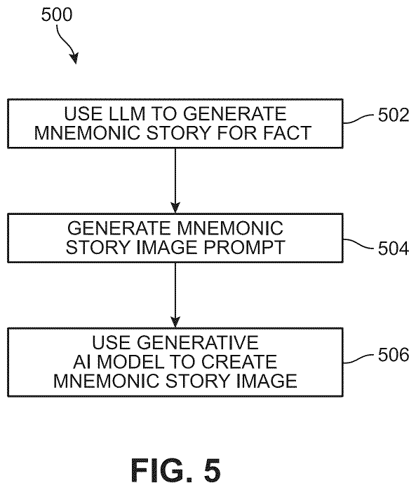

FIG. 5 is a flowchart of an example embodiment of a process 500 for creating a mnemonic story image associated with a fact for cognitive training system 100. In an example embodiment, process 500 may be implemented by components of system 100 as part of operations 204 and 206 of method 200, described above. In this embodiment, process 500 begins with an operation 502. At operation 502, a large language model, such as LLM 112, is used to generate a mnemonic story for each fact. For example, at operation 502, the prompt generated at operation 308 of process 300 may be fed into LLM 112 as an input to output the mnemonic story.

In one embodiment, standalone sentence 406 (e.g., “Abdul Hakim Sani Brown from Japan ran 100 meters in 10.05 seconds on 24 Jun. 2017.”) associated with fact 400 (shown in FIG. 4) may have an associated mnemonic story generated at operation 502. For example, the mnemonic story associated with standalone sentence 406 generated at operation 502 is “A brown bunny (‘Brown’) holding chopsticks (‘Japan’) and running”. In this case, the mnemonic story associates the brown bunny for Abdul Hakim Sani Brown, chopsticks for Japan, and running for the 100 meters. With this arrangement, these elements of the mnemonic story help reinforce the fact and the standalone sentence in the memory of the user.

Next, process 500 may proceed to an operation 504 where a mnemonic story image prompt is generated. The mnemonic story image prompt generated at operation 504 may be utilized by another component of system 100 to generate a mnemonic story image based on the mnemonic story. For example, in one embodiment, LLM 112 may generate a mnemonic story image prompt at operation 504 that is provided as an input to generative AI model 110 for generating a mnemonic story image. In an example embodiment, the mnemonic story image prompt may include instructions, limitations, requirements, and/or rules for generating a mnemonic story image to visualize or illustrate the elements of the mnemonic story. In some cases, the prompt may require that the mnemonic story image represent at least one element of the mnemonic story, be a visual scene that is easily drawn and understood, and be concise.

The mnemonic story image prompt generated at operation 504 may then be utilized as an input for an operation 506 where a generative AI model, such as generative AI model 110, is used to generate a mnemonic story image as an output. For example, generative AI model 110 may use the requirements received in the mnemonic story image prompt to generate the mnemonic story image that conforms to the instructions in the prompt.

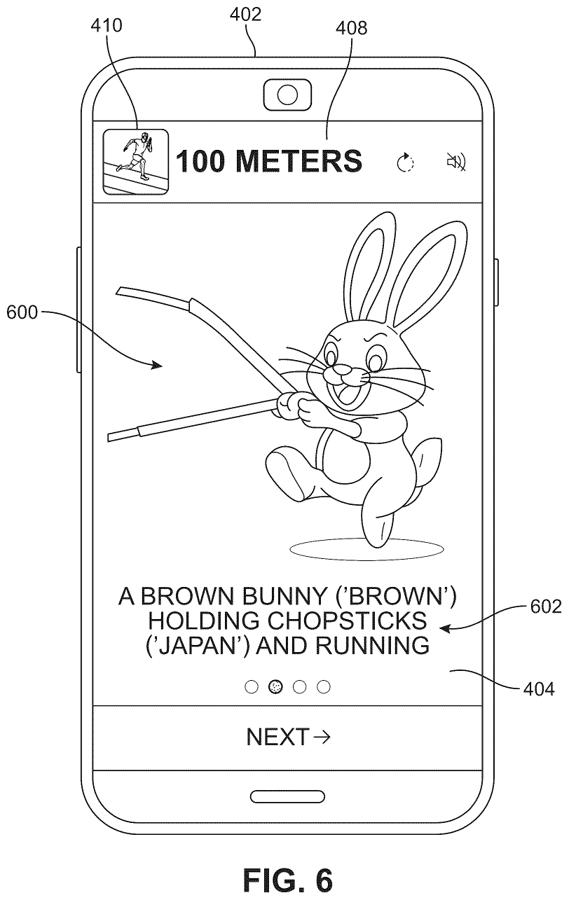

Referring now to FIG. 6, a representative view of an example embodiment of a mnemonic story image 600 associated with fact 400 is shown. In this embodiment, mnemonic story image 600 for fact 400 is shown on display 404 of smartphone 402 of a user. As shown in FIG. 6, mnemonic story image 600 is a visualization or illustration of a mnemonic story 602 that has been generated based on standalone sentence 406 for fact 400 (as shown in FIG. 4). In this case, mnemonic story 602 (e.g., “A brown bunny (‘Brown’) holding chopsticks (‘Japan’) and running”) is visualized or illustrated in mnemonic story image 600 by showing a depiction or image of a brown bunny holding chopsticks and running. In an example embodiment, mnemonic story 602 and mnemonic story image 600 may be generated as part of operations 502 and 506 of process 500, described above.

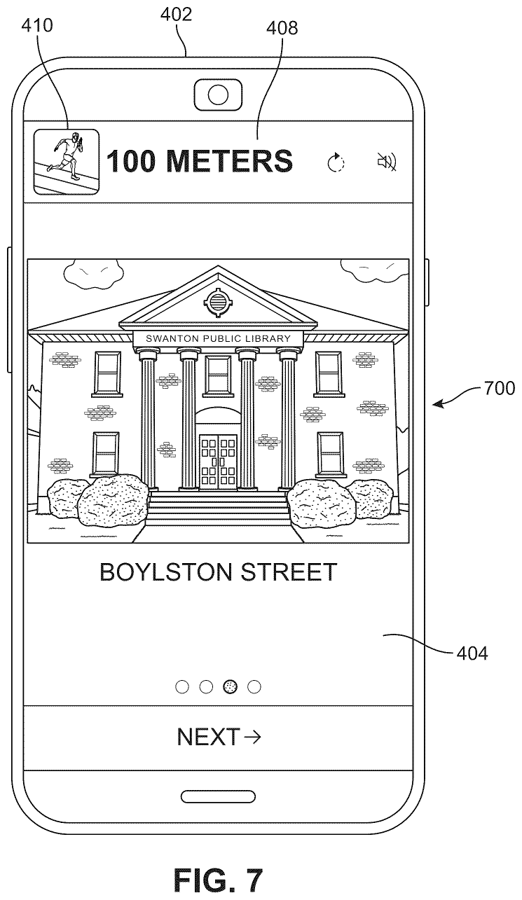

FIG. 7 shows an example embodiment of an image of a place 700 associated with a fact for cognitive training system 100. In some embodiments, a place at a location is associated with the fact, the standalone sentence, the mnemonic story, and the mnemonic story image. Associating a place, such as a building or façade, at a location helps a user embed the fact in their memory. In some cases, the place selected may be from a location within a geographic area known to the user or near a location of the user. For example, a user's location may be obtained from user device 130, such as smartphone 402. In other cases, the place may be selected from a location within a geographic area picked or specified by the user. In still other cases, the place may be selected from a location within a random geographic area.

In this embodiment, place 700 is a library located on Boylston Street within a predetermined geographic area of the user's location (e.g., a home address, a location specified by the user during registration with system 100, or a location determined obtained from user device 130). For example, the predetermined geographic area may be approximately 5-10 miles of the current location of the user. As shown in FIG. 7, place 700 is displayed to the user on display 404 of smartphone 402. In some embodiments, a user may have a place queue that is generated based on the user's location and that is populated with a plurality of places within the predetermined geographic area of the user's home location. Each of the plurality of places are different from each other so that place 700 shown to the user is uniquely associated to a fact for that user. That is, while different users within a similar home location may share some of the same places and locations in their queues, the association between the fact and the place will be unique for each user.

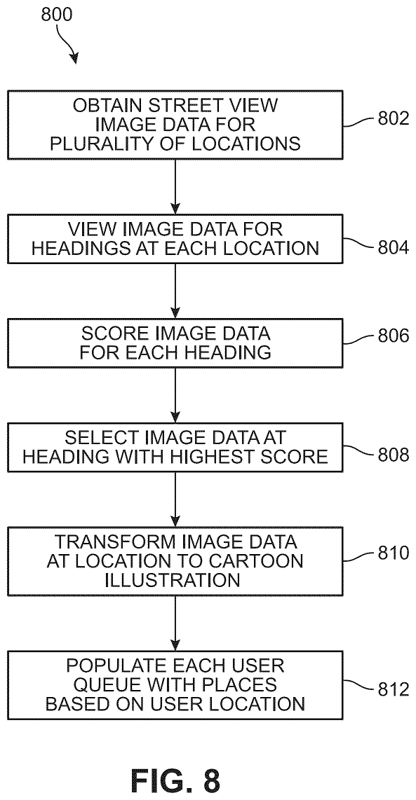

Referring now to FIG. 8, a flowchart of an example embodiment of a process 800 for selecting locations and places to be used as part of cognitive training system 100 is shown. In an example embodiment, process 800 may be implemented by components of system 100 as part of operation 208 of method 200, described above. In this embodiment, process 800 begins with an operation 802. At operation 802, street view image data for a plurality of locations is obtained. For example, street view image data for the plurality of locations may be obtained from GIS 118. In various embodiments, system 100 may obtain data associated with the 2644 most populated geographic areas (e.g., cities, towns, etc.) and create a grid with areas approximately 100 square meters apart for each of the geographic areas. Coordinates for each area within the grid may be obtained from GIS 118. Operation 802 of process 800 uses these coordinates to obtain street view image data at each location within the grid.

Next, process 800 may proceed to an operation 804 where image data for a plurality of headings at each location is viewed by system 100. For example, at operation 804, the plurality of headings may be six different headings separated by 60 degrees (i.e., for a total of 360 degree view). The image data associated with each heading of the plurality of headings is viewed by image segmentation model 120 of system 100 which uses machine vision techniques and algorithms to analyze the image data for each heading and detect any buildings or façades present in the image. It should be understood that six headings (e.g., 60 degrees apart for a total of 360 degrees) at each location is merely exemplary, in other embodiments, the headings may be a larger or smaller number. In one embodiment, the separation between the view of each heading may be determined by dividing 360 degrees by the number of headings (n).

At an operation 806, the image data for each heading from operation 804 is scored. For example, at operation 806, image segmentation model 120 may use machine vision techniques and algorithms to score each image for the plurality of headings based on the presence or absence of a building or façade in the image. That is, image segmentation model 120 detects and assigns a value to images that contain buildings or façades at the location associated with each heading. Next, process 800 proceeds to an operation 808 where the heading at the location having the highest score is selected as the place image for that location. At operation 808, image segmentation model 120 scores the place images for each heading at the location to determine which image has the highest score for a building or façade presence (i.e., greatest amount of building or façade shown in the place image). The place image at the heading for the location having the highest score is selected at operation 808 as the place image for that location. For example, in an example embodiment, place 700 shown in FIG. 7 had the highest score for the location (e.g., Boylston Street) and is selected as the place image for that location as part of operation 808.

In some embodiments, the place image of a location is transformed into a cartoon place illustration at an operation 810 of process 800. As described above in reference to operation 210, the cartoon place illustration typically includes simplified or exaggerated features or characteristics of the photograph of the place image that is assigned to the fact so that it is more recognizable or memorable for the user than the place image. In an example embodiment, the place image may be transformed into a cartoon illustration using cartoon rendering module 122 of system 100.

Next, process 800 includes an operation 812. At operation 812, each user queue is populated with a plurality of places based on the user's location. In some embodiments, operation 812 may include pre-populating a user queue with 100 places, with each place at a different location within the predetermined geographic area of the user's home location, as described above. Additional places may be added to the user's queue once a predetermined number of facts have been assigned to places. In other embodiments, the user's queue may be populated using places selected by the user or randomly assigned to the user by system 100.

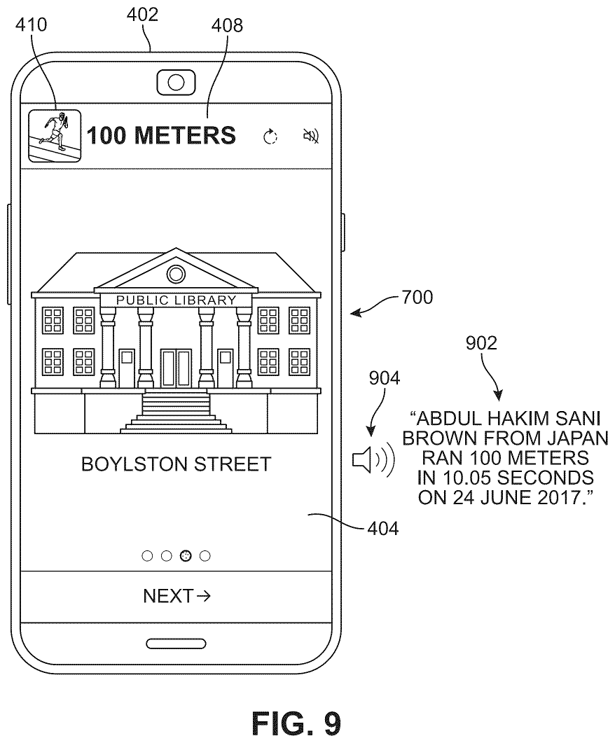

Referring now to FIG. 9, an example embodiment of a cartoon illustration 900 of a place associated with a fact and an audio presentation of the fact is shown. In this embodiment, place image 700 (as shown in FIG. 7) has been “cartoonified” into cartoon illustration 900 as part of operation 210 of method 200 and/or operation 810 of process 800 using cartoon rendering module 122 of system 100, described above. As shown in FIG. 9, place image 700 of the library located on Boylston Street has been transformed by cartoon rendering module 122 into cartoon illustration 900 of a library. In this embodiment, cartoon illustration 900 simplifies and omits extraneous elements from place image 700 so that cartoon illustration 900 is more memorable to the user.

In some embodiments, the audio that has been generated for the standalone sentence and/or the mnemonic story as part of operation 212 of method 200 may be output to the user along with cartoon illustration 900. For example, as shown in FIG. 9, cartoon illustration 900 is visible on display 404 of smartphone 402 and an audio output 902 of the standalone sentence associated with a fact (e.g., standalone sentence 406 for fact 400) is provided to the user through a speaker 904 of smartphone 402. With this arrangement, the user may associate cartoon illustration 900 of the place at the location with standalone sentence 406 and fact 400.

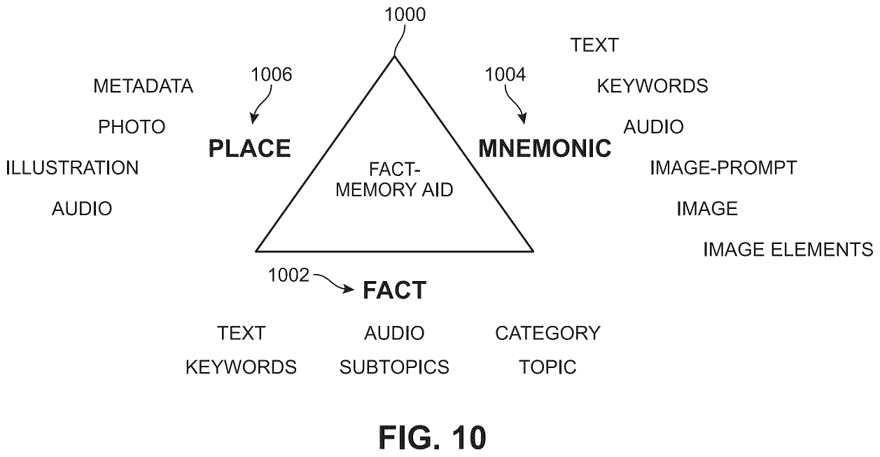

As described above in reference to method 200, cognitive training system 100 generates a fact-memory aid at operation 214 to help a user remember and recall a fact by association with a mnemonic story, a mnemonic story image, and a place at a location. FIG. 10 illustrates components associated with an example embodiment of a fact-memory aid 1000 to be used as part of cognitive training system 100. As shown in FIG. 10, fact-memory aid 1000 includes three main components, including a fact component 1002, a mnemonic component 1004, and a place component 1006. Taken together, fact component 1002, mnemonic component 1004, and place component 1006 characterize fact-memory aid 1000.

In some embodiments, fact component 1002 may include one or more factors associated with the fact, including but not limited to text (e.g., the standalone sentence), audio (e.g., the audio output), a category, a topic, one or more subtopics, and/or keywords related to the fact. In some embodiments, mnemonic component 1004 may include one or more factors associated with the mnemonic associated with the fact, including but not limited to text (e.g., the mnemonic story), audio (e.g., the audio output), the image prompt, an image (e.g., the mnemonic story image), image elements of the mnemonic image, and/or keywords related to the mnemonic story or mnemonic story image. In some embodiments, place component 1006 may include one or more factors associated with the place associated with the fact, including but not limited to audio (e.g., the audio output of the location), a place photo (e.g., place image data), an illustration (e.g., the cartoon place illustration), and/or metadata related to the place (e.g., geographical coordinates, heading, location, geographic area, etc.). With this arrangement, fact component 1002, mnemonic component 1004, and place component 1006 provide memory associations for a user to remember and recall a fact by using fact-memory aid 1000.

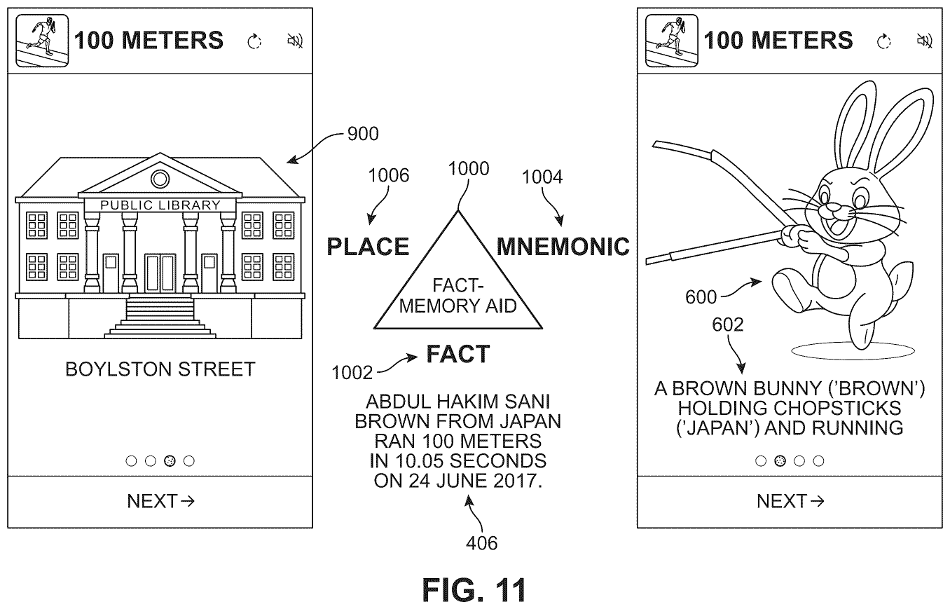

FIG. 11 is a representative view of an example embodiment of fact-memory aid 1000 to be used as part of the cognitive training system 100. In this embodiment, fact-memory aid 1000 is shown with components associated with the present embodiments described above. For example, as shown in FIG. 11, fact component 1002 includes standalone sentence 406 (e.g., “Abdul Hakim Sani Brown from Japan ran 100 meters in 10.05 seconds on 24 Jun. 2017.”) associated with fact 400 (shown in FIG. 4). Mnemonic component 1004 includes mnemonic story 602 (e.g., “A brown bunny (‘Brown’) holding chopsticks (‘Japan’) and running”) which is visualized or illustrated in mnemonic story image 600 by showing a depiction of a brown bunny holding chopsticks and running. Place component 1006 includes cartoon place illustration 900 of a library at a location (e.g., Boylston Street), which was transformed from place image 700 of the library (shown in FIG. 7). Together, fact component 1002, mnemonic component 1004, and place component 1006 provide memory associations for a user to remember and recall the details of fact 400 by using fact-memory aid 1000.

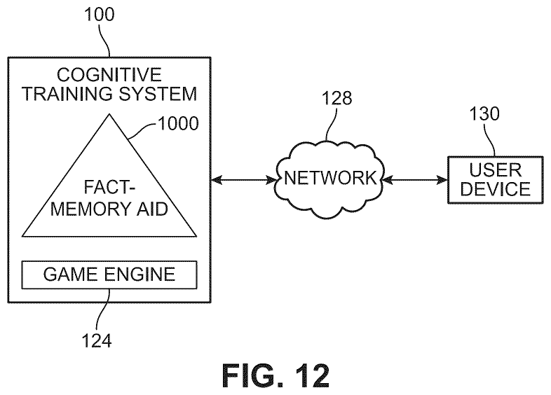

In some embodiments, a user of system 100 may participate in a game experience by playing one or more games based on fact-memory aid 1000 in order to assist the user with remembering and recalling the details of fact 400 represented by fact-memory aid 1000. Referring now to FIG. 12, a schematic view of game engine 124 of cognitive memory system 100 for providing a game experience to a user is shown. In this embodiment, a user may participate in a game experience generated by game engine 124 of cognitive training system 100 over network 128. In an example embodiment, the game experience allows a user to play one or more games based on each fact-memory aid, such as fact-memory aid 1000. By participating in the game experience generated by game engine 124 of system 100, the user practices cognitive training to help remember and recall the components of each fact-memory aid. For example, while fact-memory aid 1000 has been used as an example in the present embodiments, a plurality of fact-memory aids may be generated for a user and each may be part of a game experience to help reinforce the components of each fact-memory aid.

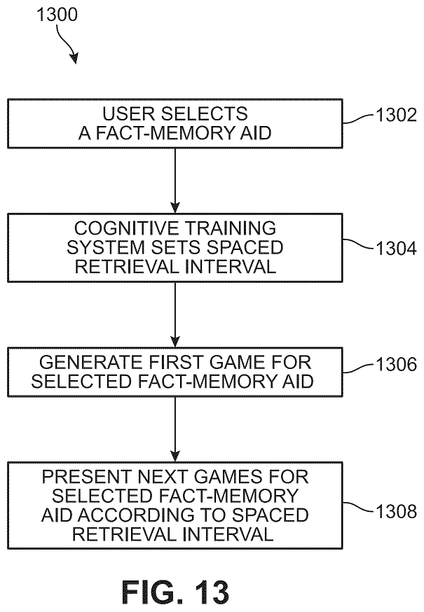

FIG. 13 is a flowchart of an example embodiment of a process 1300 for providing a game experience to a user as part of cognitive memory system 100. In this embodiment, process 1300 begins when a user selects a fact-memory aid. For example, a user may select fact-memory aid 1000 through user device 130 to begin process 1300 for a game experience. In an example embodiment, the user may swipe on a fact or standalone sentence of a fact displayed on a touch screen of user device 130 to begin process 1300. Next, process 1300 proceeds to an operation 1304. At operation 1304, cognitive training system 100 sets a spaced retrieval interval associated with the game experience for the fact-memory aid selected at operation 1302. The spaced retrieval interval is a predetermined schedule or sequence of games that are to be played by the user over a set time period to help reinforce and remember the details of the selected fact-memory aid.

For example, in one embodiment, the spaced retrieval interval for the game experience includes 8 days of games to be played spread over a 60-day time period. The spacing intervals between the days on which games are to be played varies in a way that helps anchor the details of the fact-memory aid early using shorter intervals and then reinforces the details of the fact-memory aid over the remaining time period using longer intervals. In some cases, the user may select or choose different types of spaced retrieval intervals intended to assist the user remember the details of the fact-memory aid for different durations.

Next, process 1300 proceeds to an operation 1306 where a first game of the game experience for the user is generated based on the selected fact-memory aid. For example, the first game at operation 1306 is generated by game engine 124 based on the fact-memory aid selected by the user at operation 1302. The first game tests or quizzes the user on the details associated with the selected fact-memory aid. In some embodiments, a user may play a predetermined number of games associated with a specific fact-memory aid in a single day. For example, in one embodiment, a user may play up to three games on each of the days on which games are to be played over the course of the spaced retrieval interval. In some embodiments, the games may grouped into different difficulty stages which progress from easy to hard further along into the spaced retrieval interval.

After completion of the first game at operation 1306, process 1300 for the game experience proceeds to an operation 1308. At operation 1308, the next games for the selected fact-memory aid are presented to the user according to the spaced retrieval interval set at operation 1304. For example, at operation 1308, game engine 124 may present the next games for the selected fact-memory aid to the user on each day on which games are to be played according to the sequence or schedule of the spaced retrieval interval. With this arrangement, the game experience assists the user with remembering and recalling the details of the selected fact-memory aid.

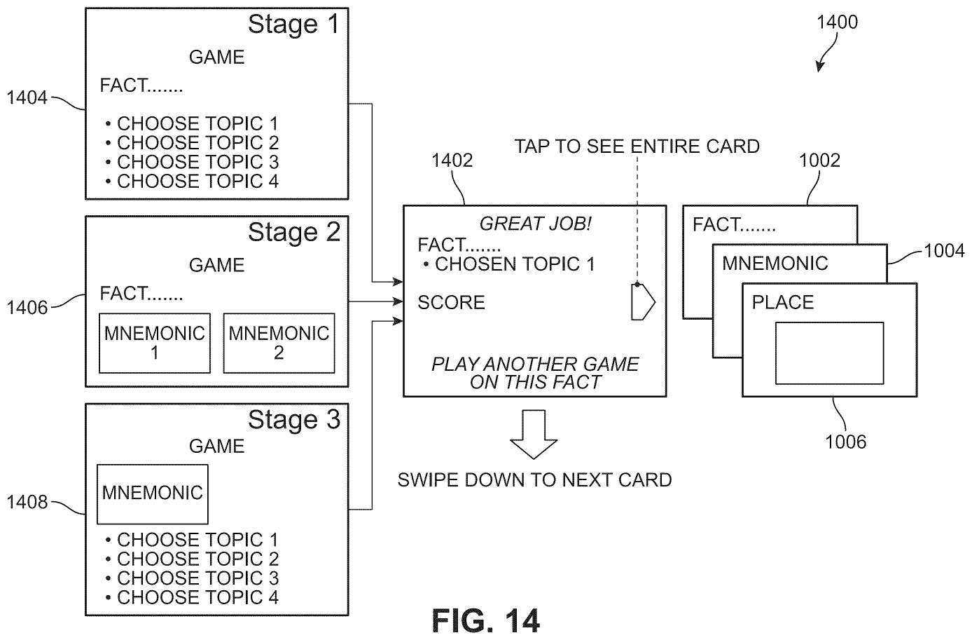

Referring now to FIG. 14, an example embodiment of a user interface 1400 for interacting with the game experience provided by game engine 124 of cognitive memory system 100 is shown. In this embodiment, user interface 1400 is presented to the user through user device 130, such as smartphone 402. A main screen 1402 shown on the display of user interface 1400 includes details associated with a fact-memory aid, such as a score for a game played by the user. The user may tap to see the components of the fact-memory aid, such as fact component 1002, mnemonic component 1004, and place component 1006 associated with fact-memory aid 1000. The user may also swipe downwards on user interface 1400 to select a next card that is associated with a different fact-memory aid.

In addition, as described above, the games of the game experience provided to the user may grouped into different difficulty stages which progress from easy to hard further along into the spaced retrieval interval. For example, as shown in FIG. 14, a first stage of games 1404 may be the easiest and include the fewest details about the fact-memory aid. Next, a second stage of games 1406 may be harder than first stage 1404 and include more details about the fact-memory aid. Finally, a third stage of games 1408 may be the hardest and include the most details about the fact-memory aid. With this arrangement, the game experience may help the user increase their knowledge and recall of the details associated with each fact-memory aid.

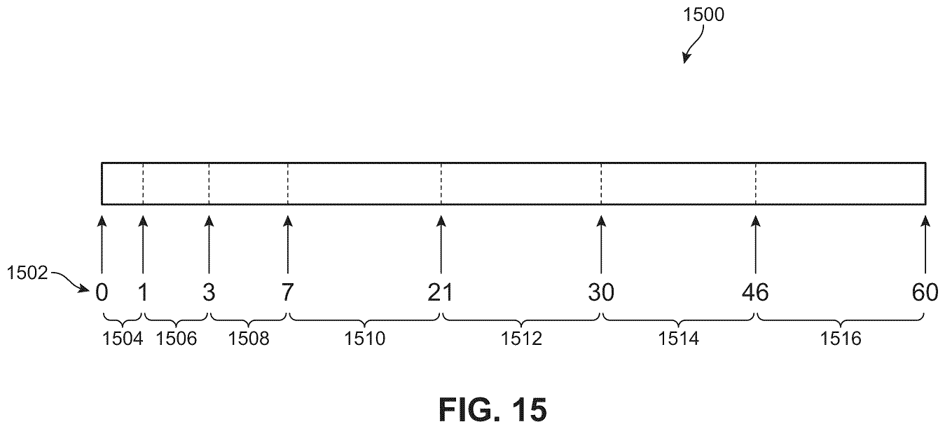

Referring now to FIG. 15, a representative view of an example embodiment of a spaced retrieval interval 1500 for a game experience associated with a fact is shown. In this embodiment, spaced retrieval interval 1500 may be set as part of operation 1304 of process 1300, described above. Spaced retrieval interval 1500 includes days 1502 on which games are to be played separated by spaced time intervals between each day on which games are to be played. In one example, spaced retrieval interval 1500 includes games on the day the user selects the fact-memory aid (i.e., day 0), games on the next day (i.e., day 1), followed by games on the third day (i.e., day 3), and the seventh day (i.e., day 7). Then, the remaining games are spaced apart by longer intervals, including games on the twenty-first day (i.e., day 21), the thirtieth day (i.e., day 30), the forty-sixth day (i.e., day 46), and the sixtieth day (i.e., day 60).

For example, in this embodiment, a first time interval 1504 between day 0 and day 1 is one day, a second time interval 1506 between day 1 and day 3 is two days, a third time interval 1508 between day 3 and day 7 is four days, a fourth time interval 1510 between day 7 and day 21 is fourteen days, a fifth time interval 1512 between day 21 and day 30 is nine days, a sixth time interval 1514 between day 30 and day 46 is sixteen days, and a seventh time interval 1516 between day 46 and day 60 is fourteen days. As can be seen in FIG. 15, the days on which games are to be played start with smaller time intervals, such as first time interval 1504, second time interval 1506, and third time interval 1508, which are on the order of several days and then extend to longer time intervals, such as fourth time interval 1510, fifth time interval 1512, sixth time interval 1514, and seventh time interval 1516, which are on the order of more than one week and up to two weeks long. With this arrangement, spaced retrieval interval 1500 for the game experience performed by the user reinforces the details associated with the fact-memory aid in the user's memory and helps the user with cognitive recall of those details.

In an example embodiment, spaced retrieval interval 1500 shown in FIG. 15 may be used to allow a user to retain the details of a fact-memory aid for long term cognitive recall. However, in other cases, a user may only need or want to retain the details of a fact-memory aid for a limited duration. For example, a user studying for an exam or test may only want to remember and recall the details of fact-memory aids relevant to the exam or test for a few months. In such cases, the user may select a different spaced retrieval interval that has a different amount of days on which games are played and/or has different spacing intervals between the days on which games are to be played. For example, a sequence of shorter spaced intervals between days on which games are played and/or fewer days on which games are played may help a user to remember and recall the details of the fact-memory aid for a few months, which is sufficient cognitive recall in order to take the exam or test, but not for long-term recall. It should be understood that other spaced retrieval intervals may be used with different numbers of days and/or different variations in spacing intervals for game experiences in accordance with the techniques described herein.

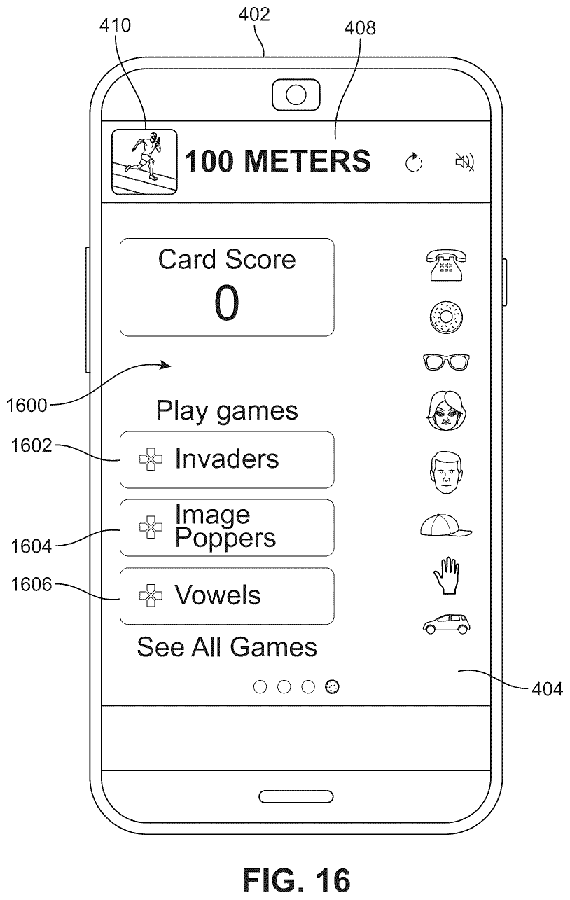

FIG. 16 illustrates an example embodiment of a game home screen 1600 for the game experience associated with a fact. In this embodiment, game home screen 1600 may be provided to the user through a user interface (e.g., user interface 1400) of a user device, such as on display 404 of smartphone 402. In an example embodiment, game home screen 1600 provides the user with available games associated with a fact-memory aid that the user may play. For example, the games available on game home screen 1600 may be determined according to spaced retrieval interval 1500, described above.

In this embodiment, game home screen 1600 includes a first game 1602 (e.g., Invaders), a second game 1604 (e.g., Image Poppers), and a third game 1606 (e.g., Vowels). As described above, on a day on which a game may be played, a user may play up to three games for a given fact-memory aid. Additionally, a user may have a cumulative card score that reflects completion of games associated with each fact-memory aid. In some embodiments, various icons or graphics (e.g., shown on the right of display 404) may be awarded to a user based on their card score for the games associated with a fact-memory aid. With this arrangement, returning or frequent users of system 100 may attain scores that reflect their continued use of system 100.

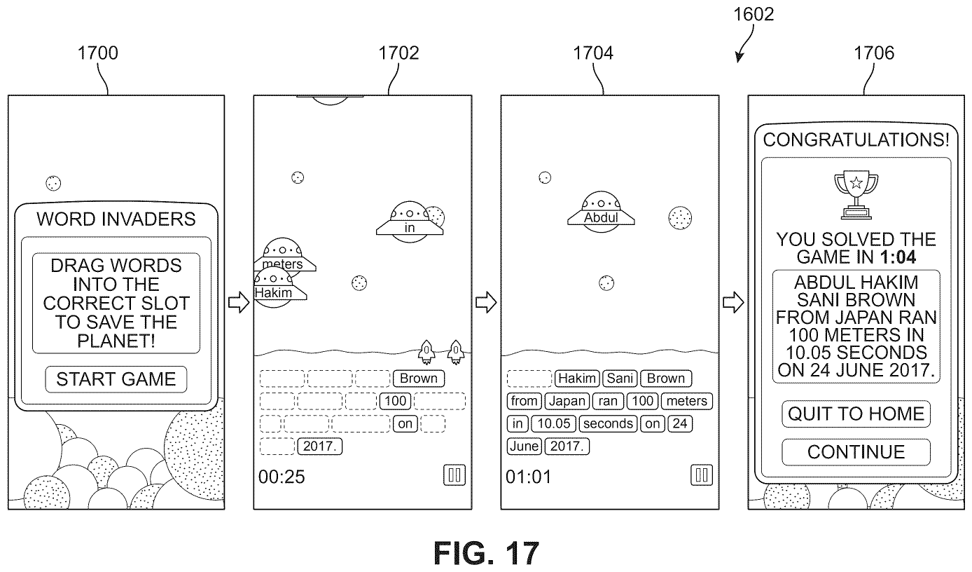

Referring now to FIG. 17, an example embodiment of first game 1602 of the game experience for cognitive memory system 100 is shown. In this embodiment, first game 1602 (e.g., Invaders) may be associated with first stage of games 1404 and may be the easiest and/or include the fewest details about the fact-memory aid. For example, in this embodiment, first game 1602 is associated with fact-memory aid 1000 and includes the words of standalone sentence 406 for fact 400 (e.g., “Abdul Hakim Sani Brown from Japan ran 100 meters in 10.05 seconds on 24 Jun. 2017.”).

As shown in FIG. 17, a first screen 1700 of first game 1602 includes the name of the game and the rules for the game to the user. A second screen 1702 and a third screen 1704 of first game 1602 show objects containing the individual words making up standalone sentence 406 that the user has to place in the correct order to re-create the sentence. Upon completion of first game 1602, the user is shown fourth screen 1706 that restates the objective of the game, completion of standalone sentence 406, and displays the completion time to the user. With this arrangement, first game 1602 of the game experience allows the user to practice cognitive recall of the details of standalone sentence 406 for fact 400.

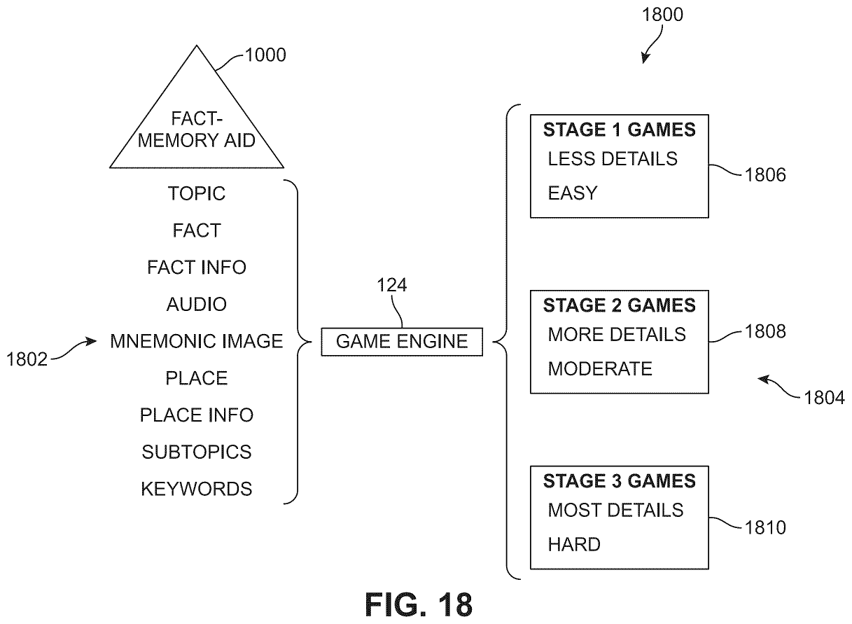

FIG. 18 illustrates a schematic view of an example embodiment of a process 1800 by game engine 124 using inputs and outputs to generate the game experience of cognitive memory system 100. In this embodiment, process 1800 implemented by game engine 124 includes a plurality of inputs 1802 that are associated with a fact-memory aid, such as fact-memory aid 1000. Plurality of inputs 1802 include details associated with fact-memory aid 1000, including, for example, a topic, a fact, fact information, audio, a mnemonic image, a place, place information, subtopics, and/or keywords.

In an example embodiment, game engine 124 may generate a plurality of games 1804 associated with fact-memory aid 1000 as an output based on plurality of inputs 1802. As shown in FIG. 18, game engine 124 generates plurality of games 1804 that includes multiple levels or stages of games having varying amounts of details and/or varying levels of difficulty. For example, in this embodiment, plurality of games 1804 includes a first stage of games 1806 that includes games using less details (e.g., from plurality of inputs 1802) associated with fact-memory aid 1000 and that have a difficulty level that is easy for a user to complete. Plurality of games 1804 also includes a second stage of games 1808 that includes games having more details than first stage 1806 and that have a moderate difficulty level. A third stage of games 1810 includes games having the most amount of details (e.g., in comparison with first stage 1806 and second stage 1808) and which games have a hard difficulty level.

With this arrangement, game engine 124 may use plurality of inputs 1802 associated with fact-memory aid 1000 to generate plurality of games 1804 associated with different stages (e.g., first stage 1806, second stage 1808, and third stage 1810) with varying amounts of details and/or varying levels of difficulty to challenge a user and help them remember and recall details associated with fact-memory aid 1000.

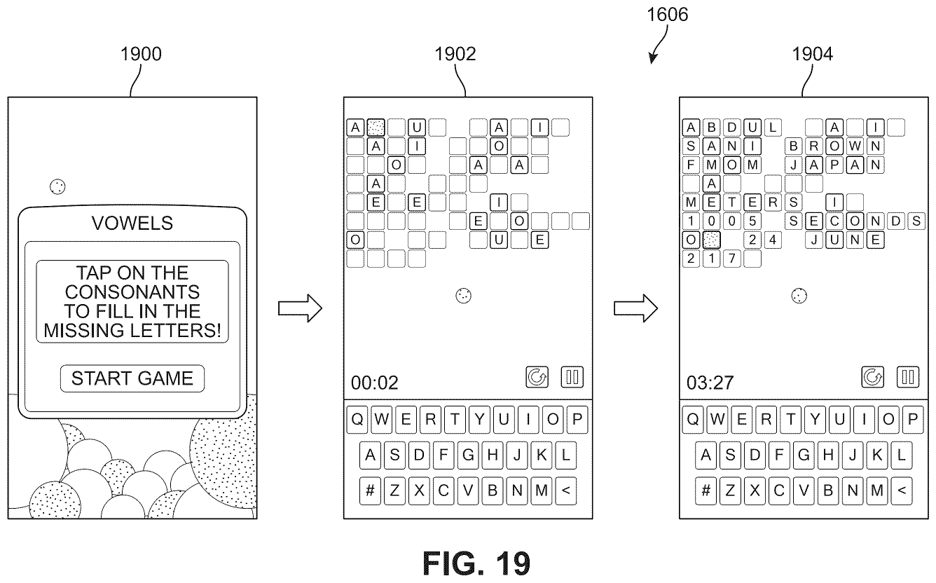

Referring now to FIG. 19, an example embodiment of another game (e.g., third game 1606) of the game experience for cognitive memory system 100 is shown. In this embodiment, third game 1606 (e.g., Vowels) may be associated with third stage of games 1810 and may be the hardest and/or include the most amount of details about the fact-memory aid. For example, in this embodiment, third game 1606 is associated with fact-memory aid 1000 and includes filling in each character making up the words and details of standalone sentence 406 for fact 400 (e.g., “Abdul Hakim Sani Brown from Japan ran 100 meters in 10.05 seconds on 24 Jun. 2017.”).

As shown in FIG. 19, a first screen 1900 of third game 1606 includes the name of the game and the rules for the game to the user. A second screen 1902 of and a third screen 1904 of third game 1606 show blank spaces for the letters and numbers making up each individual word and numbers of standalone sentence 406 that the user has to place in the correct order to re-create the sentence. With this arrangement, third game 1606 of the game experience allows the user to practice cognitive recall of the details of standalone sentence 406 for fact 400 at a level of difficulty that is greater than that of first game 1602 and/or including more details than first game 1602.

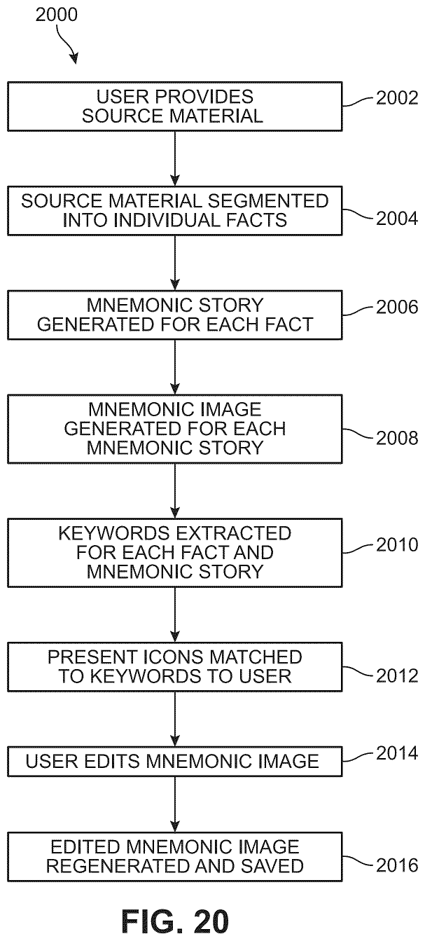

In some embodiments, a fact-memory aid may be generated based on a user's provided source material. For example, in some cases, a user may create study aids for a particular topic that the user wants to use for cognitive training. In other cases, a user may create study aids for others to use, such as a syllabus for a school course created by a professor or teacher. Referring now to FIG. 20, a flowchart of an example embodiment of a method 2000 for a user-created fact-memory aid is shown. In some embodiments, one or more operations of method 2000 may be implemented by components of cognitive training system 100 and/or user device 130.

In an example embodiment, method 2000 may begin at an operation 2002 where a user provides source material for generating a user-created fact-memory aid. In various embodiments, the user provided source material may include publications or material from the user themselves, such as articles, books, lectures, etc., or may include any source material that the user wants to use as the basis for generating a user-created fact-memory aid.

Next, method 2000 may proceed to an operation 2004. At operation 2004, the sourced material provided at operation 2002 is analyzed and segmented into individual facts. For example, at operation 2004, LLM 112 of system 100 may analyze the provided source material and segment it into one or more individual facts. Method 2000 further includes an operation 2006 where a mnemonic story is generated for each fact segmented at operation 2004 and an operation 2008 where a mnemonic story image is generated for each mnemonic story. For example, at operations 2006 and 2008, system 100 may generate a mnemonic story for each fact and a mnemonic story image for each mnemonic story according to the techniques described above with reference to operations 204 and 206 of method 200 and process 500.

In an example embodiment, method 2000 may also include an operation 2010. At operation 2010 a plurality of keywords are extracted for each fact (e.g., from operation 2004) and for each mnemonic story (e.g., from operation 2006). For example, at operation 2010, LLM 112 of system 100 may be used to extract the keywords related to the fact and mnemonic story. Next, method 2000 includes an operation 2012. At operation 2012, one or more icons, images, or emojis that are derived or matched to the keywords extracted at operation 2010 are presented to the user for their selection. For example, in some embodiments, operation 2010 may present the icons, images, or emojis to the user on a display of user device 130 for selection by the user.

Method 2000 further includes an operation 2014 where the user may edit the generated mnemonic image for each fact (e.g. which was generated at operation 2008). For example, at operation 2014, the user may select various elements of the mnemonic story image shown on the display of user device 130 to add, remove, or change. That is, operation 2014 allows the user to customize or alter the mnemonic story image generated by system 100 (e.g., by generative AI model 110) based on the source material provided by the user (e.g., at operation 2002). Next, method 2000 includes an operation 2016 where the edited mnemonic image is regenerated to incorporate the user's edits and is saved. For example, at operation 2016, system 100 may change elements of the previously-generated mnemonic story image based on the edits received from the user via user device 130 at operation 2014 in the regenerated mnemonic story image. The final, regenerated mnemonic story image may be saved in database 116 of system and be used with a place at a location to generate a user-created fact-memory aid, as described above.

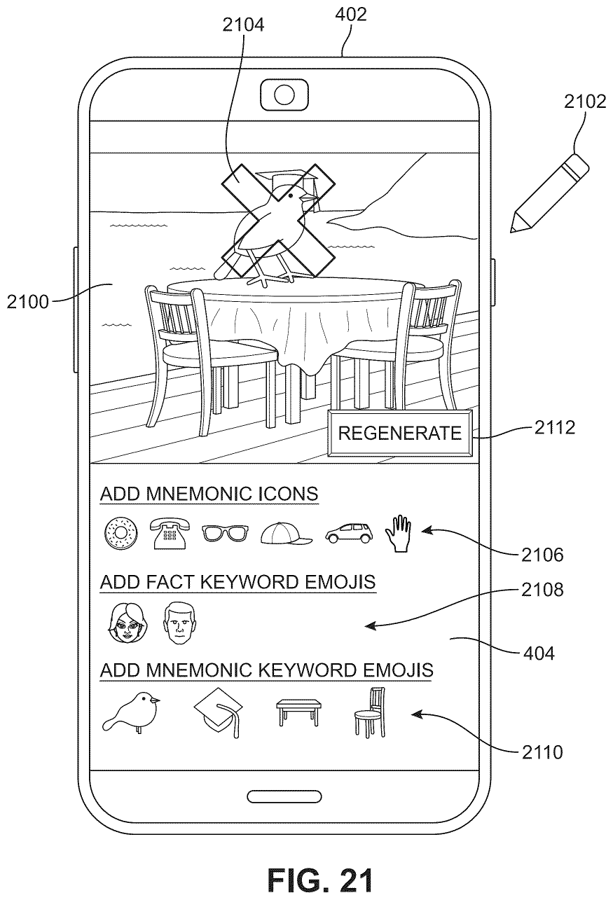

FIG. 21 is an example embodiment of a user interface for generating a user-created fact-memory aid. In this embodiment, user device 130 is in the form of smartphone 402 that allows a user to add, remove, or change elements of a mnemonic story image 2100, for example, generated as part of operation 2008 of method 2000. As shown in FIG. 21, mnemonic story image 2100 is displayed to the user on display 404 of smartphone 402 and the user may use an edit function 2102 (e.g., a pencil/edit icon) to edit mnemonic story image 2100, as described above as part of operation 2014.

For example, a user may delete an element of mnemonic story image 2100 by drawing a cross icon 2104 over elements that are to be deleted and may also choose one or more icons, images, or emojis that have been matched to keywords as part of operation 2012. As shown in FIG. 21, the user may add one or more mnemonic icons 2016, fact keyword emojis 2108, and/or mnemonic keyword emojis 2110 to mnemonic story image 2100 by using edit function 2102 to tap or select the desired element to be added. Upon completion of edits made by the user, the user may then select a regenerate option 2112 shown on display 404 of smartphone 402 to cause system 100 to regenerate mnemonic story image 2100 incorporating the changes made by the user. The regenerated mnemonic story image 2100 may then be used to create a user-generated fact-memory aid in accordance with the techniques described above.

While various embodiments of the disclosure have been described, the description is intended to be exemplary, rather than limiting and it will be apparent to those of ordinary skill in the art that many more embodiments and implementations are possible that are within the scope of the disclosure. Accordingly, the disclosure is not to be restricted except in light of the attached claims and their equivalents. Also, various modifications and changes may be made within the scope of the attached claims.

1. A method of providing cognitive training for a user implemented by a cognitive training system including at least one processor, the method comprising:
generating a standalone sentence for a fact using a large language model (LLM) of the cognitive training system;
generating a mnemonic story based on the standalone sentence using the LLM;
generating a mnemonic story image based on the mnemonic story using a generative artificial intelligence (AI) model of the cognitive training system;
assigning a place at a location to the fact, based on an input received from a user through a user device in communication with the cognitive training system;
rendering a cartoon illustration of the place using a cartoon rendering module of the cognitive training system; and
generating, by the at least one processer, a fact-memory aid associated with the fact that includes a fact component, a mnemonic component, and a place component.

2. The method according to claim 1, wherein the fact component includes the standalone sentence, the mnemonic component includes the mnemonic story image, and the place component includes the cartoon illustration of the place.

3. The method according to claim 1, further comprising:
selecting the location based on a geographic area associated with the user.

4. The method according to claim 1, wherein the assigned place includes a place image that includes a building or façade.

5. The method according to claim 1, wherein the cognitive training system further comprises a game engine; and
the method further comprises generating one or more games based on the fact-memory aid.

6. The method according to claim 5, wherein the game engine is configured to generate a plurality of games based on details associated with the fact-memory aid; and
wherein the plurality of games include multiple stages of games having different amounts of details associated with the fact-memory aid and varying levels of difficulty.

7. The method according to claim 6, further comprising:
setting a spaced retrieval interval for presenting games of the plurality of games to the user;
wherein the spaced retrieval interval is a predetermined schedule for presenting the games over a set time period spaced apart by a plurality of time intervals.

8. The method according to claim 7, wherein the plurality of time intervals includes at least two time intervals shorter than one week and at least two time intervals longer than one week.

9. The method according to claim 1, further comprising:
generating audio from text of the standalone sentence; and
outputting the generated audio to the user through a speaker of the user device.

10. The method according to claim 1, wherein the cognitive training system generates a plurality of fact-memory aids for a plurality of facts obtained from source material.

11. The method according to claim 10, wherein the source material is provided by the user.

12. A cognitive training system comprising:
a computing system comprising at least one processor;
a database in communication with the computing system;
a communication interface allowing communication with a user device associated with one or more users; and
storage for computer-readable instructions that, when executed by the at least one processer, cause the at least one processor to:
generate a standalone sentence for a fact;
generate a mnemonic story based on the standalone sentence;
generate a mnemonic story image based on the mnemonic story;
assign a place at a location to the fact, based on an input received from a user through the user device;
render a cartoon illustration of the place; and
generate a fact-memory aid associated with the fact that includes a fact component, a mnemonic component, and a place component.

13. The system according to claim 12, wherein the fact component includes the standalone sentence, the mnemonic component includes the mnemonic story image, and the place component includes the cartoon illustration of the place.

14. The system according to claim 12, wherein the instructions further cause the at least one processor to select the location based on a geographic area associated with the user.

15. The system according to claim 12, wherein the assigned place includes a place image that includes a building or façade.

16. The system according to claim 12, further comprising a game engine; and
wherein the game engine is configured to generate one or more games based on the fact-memory aid.

17. The system according to claim 16, wherein the game engine is configured to generate a plurality of games based on details associated with the fact-memory aid; and
wherein the plurality of games include multiple stages of games having different amounts of details associated with the fact-memory aid and varying levels of difficulty.

18. The system according to claim 17, wherein the instructions further cause the at least one processor to set a spaced retrieval interval for presenting games of the plurality of games to the user; and
wherein the spaced retrieval interval is a predetermined schedule for presenting the games over a set time period spaced apart by a plurality of time intervals.

19. The system according to claim 18, wherein the plurality of time intervals includes at least two time intervals shorter than one week and at least two time intervals longer than one week.

20. The system according to claim 12, wherein the cognitive training system generates a plurality of fact-memory aids for a plurality of facts obtained from source material provided by the user.
